(19) United States
(12) Patent Application Publication
(10) Pub. No.: US 2026/0308141 A1
(43) Pub. Date: Oct. 8, 2026

(54) SYNTHESIS OF ORELLANINE

(71) Applicant: ONCORENA AB, Lund (SE)

(72) Inventors: Niklas WAHLSTRÖM, Eslöv (SE); Tibor MUSIL, Södertälje (SE); Börje HARALDSSON, Varberg (SE); Pål FALCK, Oslo (NO)

(21) Appl. No.: 19/480,461

(22) PCT Filed: Apr. 30, 2024
(86) PCT No.: PCT/EP2024/061864

§ 371 Date: Oct. 31, 2025

Related U.S. Application Data
(60) Provisional application No. 63/499,787, filed on May 3, 2023.

(51) Int. Cl. C07D 401/04

(52) U.S. Cl. CPC C07D 401/04; C07B 2200/13

(57) ABSTRACT

A method for preparing Orellanine is disclosed in which Tetramethyl Orellanine is demethylated to provide Orellanine.

### FIELD OF INVENTION

The present invention relates to a method for preparing Orellanine.

### BACKGROUND OF THE INVENTION

Orellanine (see Formula I below) is a selective renal toxin occurring in relatively large amounts in several fungal species of the Cortinarius family.

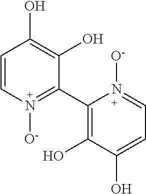

WO 2010/040750 discloses that Orellanine may be used in the treatment of renal cell carcinoma. Orellanine was taken up in human renal cancer cells and killed them with great efficiency whether they were derived from a primary tumor or from metastatic tumor tissue. The cell death progressed for many days after transient exposure to Orellanine, indicating that the toxin was actively taken up and retained by the cells.

Orellanine drug products are being developed as treatments for renal cancer. It is therefore desirable to provide a safe and efficient synthetic route to Orellanine.

Tiecco et al (Tetrahedron Vol. 42, No. 5, pages 1475 to 1485) disclose a synthetic route to Orellanine. 3-hydroxy pyridine is converted to 2-bromo-3,4-dimethoxy pyridine in a multi-step process. 2-bromo-3,4-dimethoxy pyridine is converted to Orellanine via steps of: a coupling reaction mediated by a nickel-phosphine complex; an oxidation in the presence of excess m-chloro perbenzoic acid in chloroform; and a demethylation effected with 48% hydrobromic acid at 120° C. for 5 hours.

The present inventors have sought to provide an improved synthetic route to Orellanine. In particular, the present inventors have sought to provide a synthetic route that is safe, that provides high yields, and that avoids the need for complex purification steps.

### SUMMARY OF THE INVENTION

Accordingly, the present invention provides a method for preparing Orellanine, comprising a step of:
reacting a compound of formula (II) in the presence of pyridinium hydrochloride to provide Orellanine.

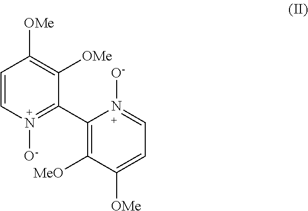

The inventors have found that reaction of the compound of formula (II) in the presence of pyridinium hydrochloride provides Orellanine in high yield and purity. This reaction also avoids the gas evolution that is experienced in prior art processes when hydrobromic acid is used as the deprotection agent.

The present invention further provides a compound of formula (III):

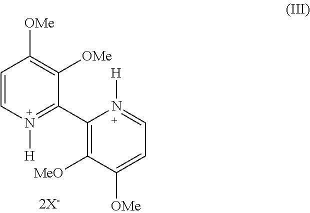

wherein X⁻ is a negatively-charged counterion.

The compound of formula (III) is an intermediate that is prepared during a purification step in an embodiment of the synthetic route to Orellanine.

### BRIEF DESCRIPTION OF THE FIGURES

FIG. 1 shows a synthetic route from 2-bromo-3,4-dimethoxy pyridine to Orellanine.

### DETAILED DESCRIPTION OF THE INVENTION

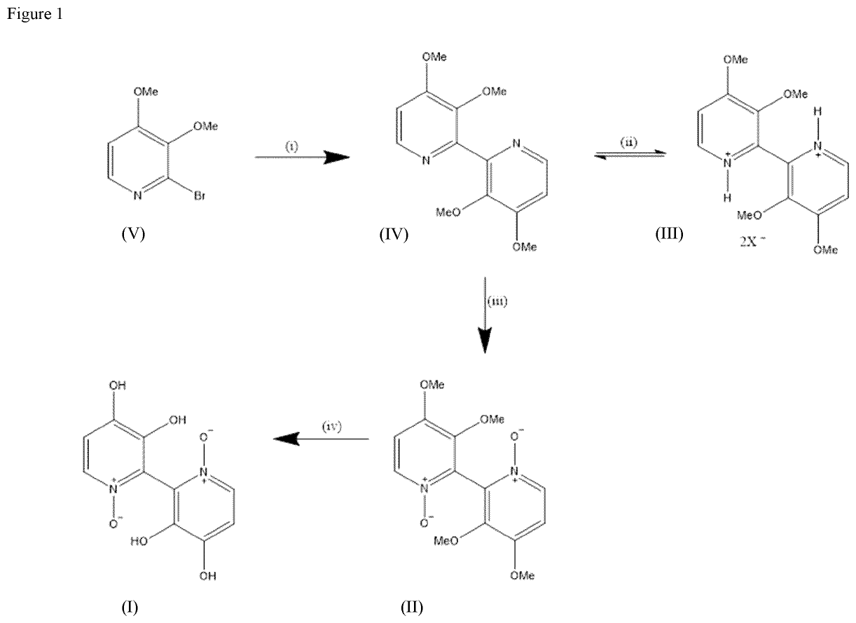

FIG. 1 shows a synthetic route to Orellanine. There are four reaction steps:
(i) coupling of 2-bromo-3,4-dimethoxy pyridine (compound of formula V) to provide tetramethyl Orelline (compound of formula IV)
(ii) purification of tetramethyl Orelline (compound of formula IV) by reversible conversion to a salt of tetramethyl Orelline (compound of formula III)
(iii) oxidation of tetramethyl Orelline (compound of formula IV) to provide tetramethyl Orellanine (compound of formula II)
(iv) demethylation of tetramethyl Orellanine (compound of formula II) to provide Orellanine (compound of formula I)

These reaction steps are described in further detail below:

### Coupling of 2-Bromo-3,4-Dimethoxy Pyridine (Compound of Formula V) to Provide Tetramethyl Orelline (Compound of Formula IV)

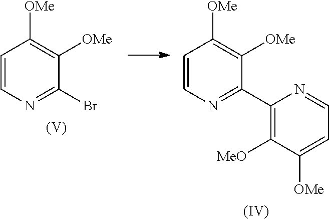

In an embodiment, the coupling is carried out in the presence of nickel (II) chloride, zinc and triphenylphosphine. The nickel (II) chloride may be anhydrous nickel (II) chloride. Suitable solvents include dimethylformamide (DMF) or a mixture of tetrahydrofuran (THF) and DMF. The reaction may be carried out at a temperature of about 60-75° C., under reflux conditions.

The amount of nickel (II) chloride may be at least 0.2 equivalents and may be approximately 1 equivalents to achieve full conversion.

The zinc may be highly activated zinc flakes (e.g. of size-325 mesh). The zinc may be present in an excess, such as at least 1 equivalent, or at least 3 equivalents or about 5.2 equivalents.

The reaction may suitably be carried out under inert conditions, such as a nitrogen atmosphere or an argon atmosphere.

In an embodiment the nickel complex is formed by a method in which triphenylphosphine is charged to the reaction vessel, followed by zinc and then nickel chloride. The emergence of a deep red/brown colour may indicate that the correct nickel complex has been formed.

In an alternative embodiment, the coupling of 2-bromo-3,4-dimethoxy pyridine (compound of formula V) is carried out in the presence of a palladium (II) acetate catalyst. For example, the coupling can be carried out in the presence of 15% Pd(OAc) 2, N—Bu₄NI, EtNiPr₂, and a DMF solvent.

In an alternative to coupling of 2-bromo-3,4-dimethoxy pyridine, tetramethyl Orelline (compound of formula IV) may be prepared by coupling of 2-iodo-3,4-dimethoxy pyridine using the same reagents and conditions as for 2-bromo-3,4-dimethoxy pyridine.

Purification of Tetramethyl Orelline (Compound of Formula IV) by Reversible Conversion to a salt of tetramethyl Orelline (compound of formula III)

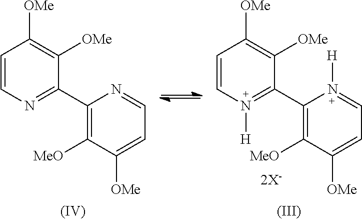

The tetramethyl Orelline (compound of formula IV) may be purified by methods known to the skilled person. The tetramethyl Orelline (compound of formula IV) may contain residues of zinc and nickel from the coupling step and it is desirable to reduce the levels of zinc and nickel, desirably to a level that is suitable for use as a pharmaceutical ingredient, e.g. 3 ppm or less for nickel and 130 ppm or less for zinc. Extractive and chromatographic approaches may be used.

In one embodiment the tetramethyl Orelline (compound of formula IV) is purified by a method wherein tetramethyl Orelline (compound of formula IV) is converted to a salt of tetramethyl Orelline (compound of formula III). The salt may be subsequently converted back to tetramethyl Orelline (compound of formula IV) as a simplified purification step for tetramethyl Orelline (compound of formula IV). With this approach it may be that chromatographic purification methods are not needed; the extractive method is sufficient to achieve tetramethyl Orelline (compound of formula IV) of suitable purity.

In one method according to this embodiment, the tetramethyl Orelline (compound of formula IV) is dissolved in dichloromethane at room temperature. Hydrochloric acid is added to provide tetramethyl Orelline dihydrochloride salt. The tetramethyl Orelline dihydrochloride salt is subjected to vacuum filtration with dichloromethane washing. The tetramethyl Orelline dihydrochloride salt is neutralised with a base such as sodium carbonate. The resulting tetramethyl Orelline (compound of formula IV) can be extracted into dichloromethane.

In another method according to this embodiment, the tetramethyl Orelline (compound of formula IV) is dissolved in dichloromethane at room temperature. Oxalic acid is added to provide tetramethyl Orelline dioxalate salt. The tetramethyl Orelline dioxalate salt is subjected to vacuum filtration with dichloromethane washing. The tetramethyl Orelline dioxalate salt is neutralised with a base such as sodium carbonate. The resulting tetramethyl Orelline (compound of formula IV) can be extracted into dichloromethane.

The compound of formula (III) includes a negatively-charged counterion, shown as 2X⁻. As an alternative to two counterions with a single negative charge, the counterion could be a single counterion with charge of −2, i.e. X²⁻. The negatively-charged counterion may be a halide ion, preferably chloride. The negatively-charged counterion could be a dioxalate anion.

### Oxidation of Tetramethyl Orelline (Compound of Formula IV) to Provide Tetramethyl Orellanine (Compound of Formula II)

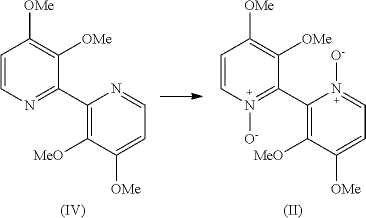

In an embodiment the oxidation is carried out in the presence of m-chloro perbenzoic acid (m-CPBA). The solvent may be dichloromethane. Other possible solvents include THE, ethyl acetate and DMF. Vacuum filtration may be used to remove byproducts.

The amount of m-CPBA may be at least 2 equivalents, or at least 3 equivalents in order to achieve fast conversion.

The tetramethyl Orellanine (compound of formula II) may be purified by adding methyl tert-butyl ether (MTBE) and crystallizing the tetramethyl Orellanine from solution. The crystals are suitably washed with MTBE and subjected to vacuum filtration.

### Demethylation of Tetramethyl Orellanine (Compound of Formula II) to Provide Orellanine (Compound of Formula I)

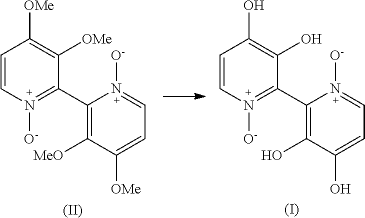

Demethylation of tetramethyl Orellanine (compound of formula II) with hydrobromic acid can provide Orellanine, but is accompanied by gas evolution. The present inventors sought to provide an alternative route to Orellanine. The present inventors surprisingly discovered that pyridinium hydrochloride provides Orellanine from tetramethyl Orellanine (compound of formula II) with high purity, thus avoiding the need for chromatographic purification, and without significant gas evolution, thus allowing for safe scale-up.

The amount of pyridinium hydrochloride may be at least 8 molar equivalents, possibly about 25 molar equivalents.

Pyridinium hydrochloride may be purchased as a reagent, or it may be prepared in situ by addition of pyridine and hydrochloric acid. In this description, the terms “pyridinium hydrochloride” and “pyridine hydrochloride” are both used to describe the reaction product of pyridine and hydrochloric acid. Another appropriate term is “pyridinium chloride”.

The temperature of the reaction may be in the range of 25-200° C., from 50-150° C., from 80-125° C., or from 95-110° C., or about 105° C. The inventors have found that effective deprotection occurs at temperatures that are lower than those typically used in a demethylation step.

Typical reaction duration may be about 1 hour, or up to 24 hours.

The solvent may be n-methyl pyrrolidone (NMP). Other possible solvents include dimethylimidazolidinone, DMF, NMP, DMA, sulfolane, and 1,4-dichlorobenzene. It is also possible to carry out the reaction in the absence of solvent.

The reaction may suitably be carried out under inert conditions, such as a nitrogen atmosphere or an argon atmosphere.

The Orellanine may be purified by methods known to the skilled person. In one method, the Orellanine is crystallized from solution and is subjected to vacuum filtration plus washing in water and ethanol. The Orellanine may then be treated with hydrochloric acid, followed by sodium carbonate. This acidification/basification may be repeated once or multiple times.

The invention will now be described by reference to examples which are not intended to be limiting of the invention:

### EXAMPLES

### Demethylation of Tetramethyl Orellanine (Compound of Formula II) to Provide Orellanine (Compound of Formula I)

### Comparative Example 1—Hydrogen Bromide with Acetic Acid

0.5 g of tetramethyl Orellanine was reacted with neat hydrogen bromide (33%) in acetic acid. The reaction took place in a sealed tube at 125° C. Orellanine was formed with about 10% impurities and an uncorrected yield of 70-85%. Scaling up this methodology would require special equipment suitable for corrosive media at high pressure.

### Comparative Example 2—Hydrogen Bromide with Acetic Acid

8 g of tetramethyl Orellanine was reacted with neat hydrogen bromide (33%) in acetic acid. To control exothermic reactions and excessive gas evolution, semi-batch synthesis was used. Slow reaction occurred under a slow flow of nitrogen. Several fresh additions of acid were required to drive conversion such that approximately 50 equivalents of hydrogen bromide were used in total. Isolation by filtration of the crude product and analysis by NMR showed that several degradation impurities formed and the content of Orellanine was about 40%. Due to the problems with gas evolution, it was concluded that this approach cannot be used at scale.

### Comparative Example 3—Boron Tribromide

Tetramethyl Orellanine was reacted with an excess of boron tribromide in dichloromethane. The temperature was controlled from −4 to 0° C. and then from 0-25° C. Degradation of the N-oxide and formation of impurities was observed. This reaction did not lead to Orellanine.

### Comparative Example 4—Hydrogen Iodide

39.5 mg tetramethyl Orellanine was reacted with hydrogen iodide in the dark in a sealed vial. No Orellanine was formed overnight at room temperature. When heated, decomposition and iodination reactions took place.

### Example 1—Pyridine Hydrochloride

22 mg tetramethyl Orellanine was reacted with a large excess of pyridine hydrochloride at 80° C. overnight. This provided a mixture of Orellanine, monodeprotected methyl Orellanine, and starting material in a ratio of 10:3:0.3.

### Example 2—Pyridine Hydrochloride

1.74 g tetramethyl Orellanine was reacted with 25 equivalents of pyridine hydrochloride in NMP. The reaction mixture was warmed from 25° C. to 105° C. The reaction mixture was cooled before addition of water to control the quench, was stirred for 30 minutes, and then was isolated by vacuum filtration through P3 filter paper. Orellanine was obtained in high purity. Residual solvents were detected as follows: NMP 3045 ppm, pyridine 5361 ppm, ethanol 817 ppm.

### Example 3—Pyridine Hydrochloride

48.05 g of Orellanine was prepared by reaction of tetramethyl Orellanine with 25 equivalents of pyridine hydrochloride in NMP. The reaction mixture was warmed from 25° C. to 105° C. The Orellanine product was treated with hydrochloric acid, followed by sodium carbonate. This acidification/basification was repeated several times. The calculated yield was 79.5%.

1. A method for preparing Orellanine, comprising a step of:
reacting a compound of formula (II) in the presence of pyridinium hydrochloride to provide Orellanine.

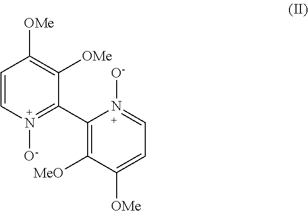

2. The method according to claim 1, wherein reacting the compound of formula (II) takes place at a temperature in the range of 80 to 125° C.

3. The method according to claim 2, wherein reacting the compound of formula (II) takes place a temperature of about 105° C.

4. The method according to claim 1, further comprising a step of:
reacting a compound of formula (IV) to provide the compound of formula (II).

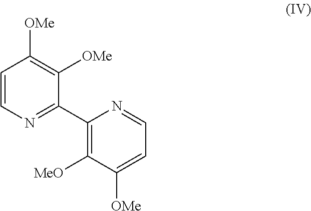 [SMILES: COc1ccnc(-c2nccc(OC)c2OC)c1OC]

5. The method according to claim 4, wherein the compound of formula (IV) is reacted with m-chloro perbenzoic acid.

6. The method according to claim 4, wherein the compound of formula (II) is purified by crystallizing the compound of formula (II) from a solution comprising methyl tert-butyl ether.

7. The method according to claim 4, further comprising a step of purifying the compound of formula IV by reversible conversion to a compound of formula III 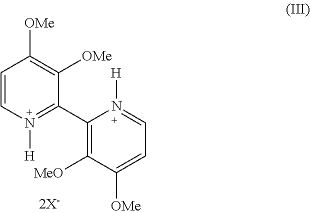
wherein X⁻ is a negatively-charged counterion.

8. The method according to claim 7, wherein the compound of formula (IV) is reacted with hydrochloric acid.

9. The method according to claim 7, wherein the compound of formula (IV) is reacted with oxalic acid.

10. The method according to claim 4, further comprising a step of:
reacting a compound of formula (V) to provide the compound of formula (IV).

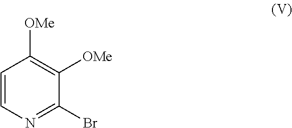 [SMILES: COc1ccnc(Br)c1OC]

11. The method according to claim 10, wherein the compound of formula (IV) is reacted in the presence of nickel (II) chloride, zinc and triphenylphosphine.

12. A compound of formula (III): 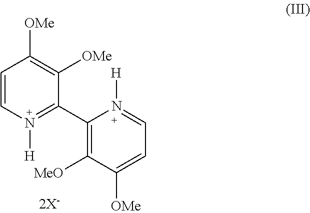
wherein X⁻ is a negatively-charged counterion.

13. The compound according to claim 12 wherein XX⁻ is a halide ion, preferably chloride.

14. The compound according to claim 12 wherein 2XX⁻ is a dioxalate anion.
